(19) United States
(12) Patent Application Publication
(10) Pub. No.: US 2014/0290328 A1
(43) Pub. Date: Oct. 2, 2014

(54) TEST SYSTEM AND METHOD

(71) Applicant: HON HAI PRECISION INDUSTRY CO., LTD., New Taipei (TW)

(72) Inventors: SHIH-CHENG WANG, New Taipei (TW); YI-WEN CAI, New Taipei (TW)

(73) Assignee: HON HAI PRECISION INDUSTRY CO., LTD., New Taipei (TW)

(21) Appl. No.: 13/952,793

(22) Filed: Jul. 29, 2013

(30) Foreign Application Priority Data
Apr. 2, 2013 (CN) 2013101133307

(51) Int. Cl. G01N 33/00

(52) U.S. Cl. CPC G01N 33/0006
USPC 73/1.06

(57) ABSTRACT

A test system and a test method adapted for a remote control device are disclosed. The remote control device is electrically connected to gas supply equipment. The gas supply equipment stores gas and is connected to a gas actuating device. A gas detector is near to the gas actuating device. The method includes: providing a test interface, acquiring a gas detector on the test interface and generating a trigger signal to start testing the gas detector; remotely controlling the gas supply equipment to generate high pressure; and controlling the gas actuating device to spew out gas. The method further includes: comparing the concentration of the gas with a stored reference concentration to determine whether or not the gas detector works properly and generating a result.

### BACKGROUND

1. Technical Field

The disclosure relates to test technology, and more particularly to a test system for testing a gas detector and a test method adapted for the system.

2. Description of Related Art

A gas detector is often positioned in a high place or other location that is not convenient for a user to access. The user must manually operate the gas detector to check whether or not the gas detector works properly, which is very inconvenient.

Therefore, what is needed is a test system and method to overcome the described shortcomings.

### BRIEF DESCRIPTION OF THE DRAWINGS

FIG. 1 is a block diagram of applying a test system in accordance with an exemplary embodiment.

FIG. 2 is a block diagram of the test system of FIG. 1.

FIG. 3 is a flowchart of a method for testing a gas detector adapted for the test system of FIG. 2.

FIG. 4 is a schematic view of a test interface for testing a gas detector in accordance with an exemplary embodiment.

### DETAILED DESCRIPTION

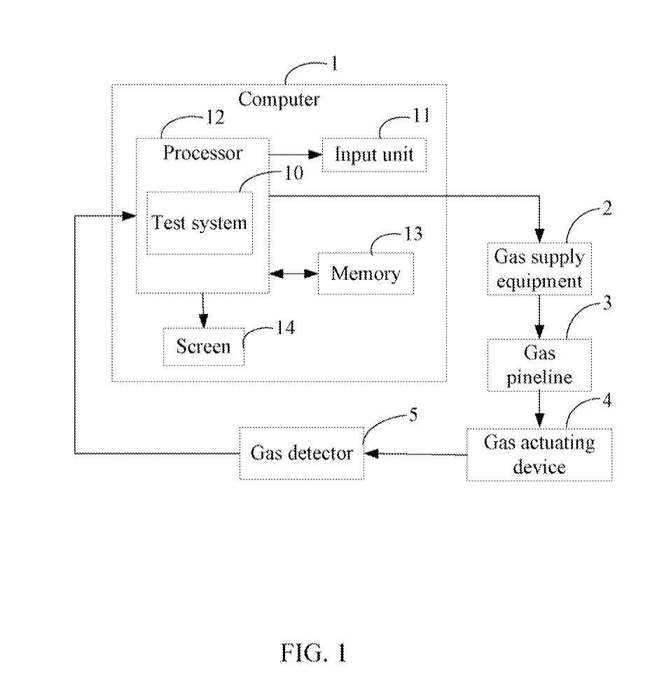

FIG. 1 is a block diagram of applying a test system 10 in accordance with an exemplary embodiment. The test system 10 is applied to a remote control device. In the embodiment, the remote control device is a computer 1. The test system 10 is utilized to generate a trigger signal to test a gas detector 5 and obtain a test result. In the embodiment, the gas detector 5 is a carbon dioxide detector.

The computer 1 includes an input unit 11, a processor 12, a memory 13, and a screen 14. The input unit 11 generates input signals in response to a user's input. The processor 12 controls the computer 1 to operate and performs the test system 10. The memory 13 stores data, such as a reference concentration of gas. The screen 14 displays information.

The computer 1 is electrically connected to gas supply equipment 2 via wiring. The gas supply equipment 2 stores gas, and the computer 1 controls the gas supply equipment 2 to release gas. A gas pipeline 3 is connected to the gas supply equipment 2 and transmits the gas from the gas supply equipment 2 to a gas actuating device 4. The gas actuating device 4 is utilized to spew out the gas from the gas pipeline 3. A gas detector 5 detects the presence of gas and measures a concentration of the detected gas.

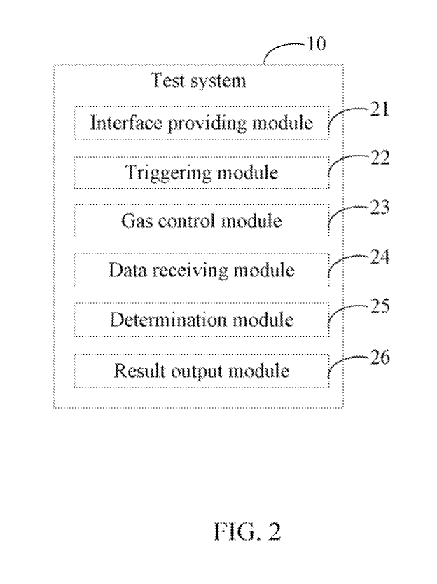

As shown in FIG. 2, the test system 10 includes an interface providing module 21, a triggering module 22, a gas control module 23, a data receiving module 24, a determining module 25, and a result output module 26, each of which is a collection of instructions executed by the processor 12.

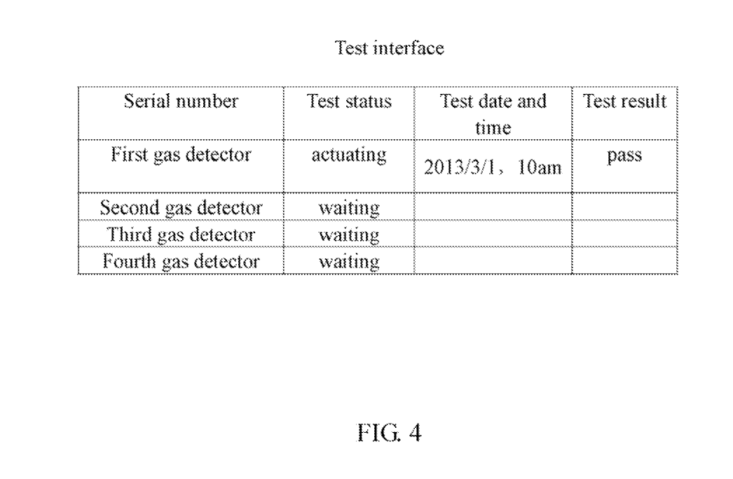

The interface providing module 21 provides a test interface. The test interface can be a touch-screen to allow a user to select a gas detector. The test interface displays several gas detectors 5. A user selects one or more gas detectors to test a concentration of gas. As shown in FIG. 4, the test interface provides four gas detectors 5 that can be tested by the test system 10. The test interface includes a serial number column, a test status column, a test date and time column, and a test result column.

The serial number column arranges the gas detectors 5 in a serial order. The test status column represents a testing state of the gas detector 5, such as an “actuating” state and a “waiting state,” for example. As shown in FIG. 4, when a first gas detector 5 is selected for testing, the first detector 5 is actuated. Gas detectors 5 that are not selected on the test interface remain in the “waiting” state. The test date and time column represents a date and time that the test system 10 tests a gas detector 5. For example, FIG. 4 shows that the first gas detector 5 was tested at 10 am on Mar. 1, 2013. The test result column represents a test result of the gas detector 5. For example, FIG. 4 shows that the first gas detector 5 passes testing.

The triggering module 22 generates a trigger signal to start testing a gas detector 5 that has been selected by a user. If the test interface only includes one gas detector, the triggering module 22 automatically selects the gas detector 5 to test. The gas detector 5 may be positioned in a room, a building, or an office, for example. The gas control module 23 remotely controls the gas supply equipment 2 to increase pressure of the selected gas in response to the trigger signal, and controls the gas to flow into the gas pipeline 3. The gas control module 23 further controls the gas actuating device 4 to spew out the gas according to a preset configuration. The preset configuration may be a preset quantity or a preset spewing direction, for example. Therefore, the gas is dispersed from the gas actuating device 4.

The gas detector 5 is near to the gas actuating device 4. The gas detector 5 measures the concentration of the gas and sends a signal containing the concentration of the gas to the data receiving module 24 of the computer 1. The data determination module 25 compares the measured concentration of the gas with the stored reference value to determine whether or not the gas detector 5 is qualified. If the measured concentration of the gas is greater than or equal to the reference value, the determination module 25 determines that the gas detector 5 passes testing; if the measured concentration of the gas is less than the reference value, the determination module 25 determines that the gas detector 5 fails testing.

The result output module 26 generates a test result of the tested gas detector 5 and outputs the test result to the screen 14 to display the test result. Therefore, a user can easily monitor the working state of several gas detectors.

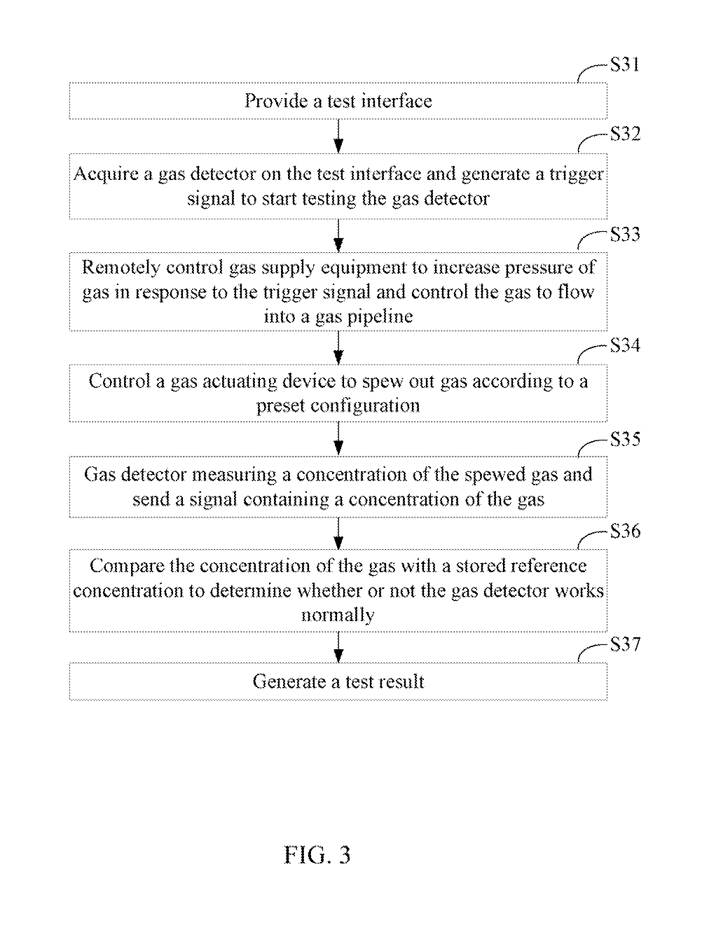

FIG. 3 is a flowchart of a method for testing a gas detector. In step S31, the interface providing module 21 provides a test interface. In step S32, the triggering module 22 generates a trigger signal to start testing a gas detector 5 in response to a user's selection. In step S33, the gas control module 23 remotely controls the gas supply equipment 2 to increase pressure of the selected gas in response to the trigger signal, and controls the gas to flow into the gas pipeline 3.

In step S34, the gas control module 23 further controls the gas actuating device 4 to spew out the gas according to a preset configuration. In step S35, the gas detector 5 measures a concentration of the spewed gas, and sends a signal containing the concentration of the gas to the data receiving module 24 of the computer 1.

In step S36, the determination module 25 compares the measured concentration of the gas with a stored reference concentration of the gas to determine whether or not the gas detector 5 works normally. In step S37, the result output module 26 generates a test result of the tested gas and controls the screen 14 to display the test result.

Although the present disclosure has been specifically described on the basis of the exemplary embodiment thereof, the disclosure is not to be construed as being limited thereto. Various changes or modifications may be made to the embodiment without departing from the scope and spirit of the disclosure.

What is claimed is:

1. A test system, wherein the test system is applied to a remote control device, the remote control device is electrically connected to gas supply equipment, the gas supply equipment stores gas and is connected to a gas actuating device for spewing out the gas, and a gas detector is near to the gas actuating device and measures a concentration of the gas, the test system comprising:
an interface providing module to provide a test interface;
a triggering module to acquire a gas detector on the test interface and generate a trigger signal to start testing the gas detector;
a gas control module to remotely control the gas supply equipment to increase pressure of the gas in response to the trigger signal and control the gas actuating device to spew out gas according to a preset configuration;
a data receiving module to receive a signal containing a measured concentration of the gas from the gas detector;
a determination module to compare the measured concentration of the gas with a stored reference concentration to determine whether or not the gas detector work normally; and
a result output module to generate a test result;
wherein the test system further comprises at least one processor to execute the modules of the test system.

2. The test system of claim 1, wherein the test interface comprises a serial number column, a test status column, a test date and time column, and a test result column, the serial number column arranges the gas detectors in a serial order, the test status column represents a testing state of the gas detector and comprises an actuating state and a waiting state, the test date and time column represents a date and time the test system tests the gas detector, and the test result column represents a test result of the gas detector.

3. The test system of claim 1, wherein the preset configuration may be a preset quantity or a preset spewing direction.

4. The test system of claim 1, wherein when the measured concentration of the gas is less than the reference concentration, the determination module determines the gas detector fails testing, and the measured concentration of the gas is greater than or equal to the reference concentration, the determination module determines that the gas detector passes testing.

5. A test method adapted for a remote control device, wherein the remote control device is electrically connected to gas supply equipment, the gas supply equipment stores gas and is connected to a gas actuating device for spewing out the gas, and a gas detector is near to the gas actuating device and measures a concentration of the gas, the test method comprising:
providing a test interface;
acquiring a gas detector on the test interface and generating a trigger signal to start testing the gas detector;
remotely controlling the gas supply equipment to increase pressure of the gas in response to the trigger signal and controlling the gas actuating device to spew out gas according to a preset configuration;
receiving a signal containing a measured concentration of the gas from the gas detector;
comparing the measured concentration of the gas with a stored reference concentration to determine whether or not the gas detector work normally; and
generating a test result.

6. The test method of claim 5, wherein the test interface comprises a serial number column, a test status column, a test date and time column, and a test result column, the serial number column arranges the gas detectors in a serial order, the test status column represents a testing state of the gas detector and comprises an actuating state and a waiting state, the test date and time column represents a date and time the test system tests the gas detector, and the test result column represents a test result of the gas detector.

7. The test method of claim 5, wherein the preset configuration may be a preset quantity or a preset spewing of direction.

8. The test method of claim 5, wherein when the measured concentration of the gas is less than the reference concentration, the gas detector fails testing, and the measured concentration of the gas is greater than or equal to the reference concentration, the gas detector passes testing.
